(19) United States
(12) Patent Application Publication
(10) Pub. No.: US 2024/0133765 A1
(43) Pub. Date: Apr. 25, 2024

(54) AIR QUANTITY ANALYZER IN UNDERGROUND COAL MINES AND ITS ANALYSIS METHOD

(71) Applicant: CCTEG Shenyang Research Institute, Fushun (CN)

(72) Inventors: Yong Sun, Fushun (CN); Yuntao Liang, Fushun (CN); Haizhu Luo, Fushun (CN); Depeng Zhang, Fushun (CN); Wenbin Feng, Fushun (CN); Junjie Zhang, Fushun (CN); Xinfeng Cui, Fushun (CN)

(21) Appl. No.: 17/973,011

(22) Filed: Oct. 24, 2022

(51) Int. Cl. G01M 3/20; G08B 21/12

(52) U.S. Cl. CPC G01M 3/20; G08B 21/12

(57) ABSTRACT

The disclosure includes an air quantity analyzer for use in underground coal mines, comprising an explosion-proof enclosure. According to some embodiments, the air quantity analyzer for use in underground coal mines comprises a gas pipeline inside of the explosion-proof enclosure, the gas pipeline coupled to a mechanical component selected from the group consisting of an air sampler, a gas pool, and combinations thereof, a detection device inside of the explosion-proof enclosure, the detection device having a vacuum pump, wherein the vacuum pump is coupled to the gas pipeline, and a central processor inside of the explosion-proof enclosure and electrically coupled to the vacuum pump of the detection device, the central processor electrically coupled to an electric component selected from the group consisting of a light source, a detector, a power conversion module, a display and alarm module, a data communication module, and combinations thereof.

### BACKGROUND

### Field

The invention relates to air quantity analysis devices for underground coal mine use. In particular, this invention relates to an analyzer for measuring air quantity and air leakage in underground coal mines and the analysis method.

### Description of Related Art

One of the primary technical means for ensuring mine safety is mine ventilation. Mine ventilation refers to the use of either mechanical or natural air pressure to continuously transport fresh air to each underground mining site that needs air, thus diluting or completely removing various toxic or otherwise harmful gases and dust. The ultimate goal for mine ventilation is to ensure good air condition for workers in their workplace.

As work on a mine wall (working face) progresses, coal rock around this working face is constantly being deformed. Consequently, the working face and the surrounding goaf become connected through fractures, thus changing the distribution of the airflow field. This reduces ventilation efficiency and air quality and may even cause oxidation-based spontaneous combustion of float coal.

To maintain a reasonable, stable, and reliable ventilation system, it is necessary to conduct thorough measurements of the quantity of mine air regularly to keep in check the mine air quantity, air velocity, and air leakage. Only through these consistent measurements can staff adjust the ventilation system in a timely fashion to regulate the air quantity to meet air quantity requirements on each air-use site.

Typically, air quantity measurement in underground coal mines is conducted manually by a dedicated operator(s) who use an anemometer to measure the air velocity and a meter rule to find the width and height of the roadway. The operator(s) would then use these hand measurements to calculate the area of the roadway and then use this area and the measured air velocity to calculate the air quantity using a well-known empirical formula. However, as with any measurements performed by hand, human error becomes a factor, and there is usually inaccuracy between the measured air quantity and the actual air quantity. Thus, there is a need for systems and methods to remedy these deficiencies found in the prior art.

### SUMMARY

The disclosure includes an air quantity analyzer for use in underground coal mines, comprising an explosion-proof enclosure. In some embodiments, the air quantity analyzer for use in underground coal mines comprises a gas pipeline inside of the explosion-proof enclosure, the gas pipeline coupled to a mechanical component selected from the group consisting of an air sampler, a gas pool, and combinations thereof. According to some embodiments, the air quantity analyzer for use in underground coal mines comprises a detection device inside of the explosion-proof enclosure, the detection device having a vacuum pump, wherein the vacuum pump is coupled to the gas pipeline. The air quantity analyzer for use in underground coal mines may comprise a central processor inside of the explosion-proof enclosure and electrically coupled to the vacuum pump of the detection device, the central processor electrically coupled to an electric component selected from the group consisting of a light source, a detector, a power conversion module, a display and alarm module, a data communication module, and combinations thereof.

In some embodiments, the air sampler further comprises a sampling head. According to some embodiments, the air sampler further comprises a current regulator coupled to the sampling head. The air sampler may further comprise a dust and moisture removal device coupled to the current regulator. In some embodiments, the air sampler further comprises a spiral gas circuit coupled to the dust and moisture removal device.

According to some embodiments, the central processor is electrically coupled to an electric component selected from the group consisting of a laser profiler, an airflow velocity sensor, and combinations thereof.

The power conversion module may further comprise at least one rechargeable lithium battery arranged and configured to supply power to an electrical component selected from the group consisting of the light source, the vacuum pump, the display and alarm module, the data communication module, the airflow velocity sensor, the laser profiler, and combinations thereof.

In some embodiments, the air quantity analyzer for use in underground coal mines comprises a second air quantity analyzer for use in underground coal mines According to some embodiments, the data communication module comprises a wired transmission module coupled to the second air quantity analyzer for use in underground coal mines. The data communication module may comprise a wireless transmission module coupled to the second air quantity analyzer for use in underground coal mines.

In some embodiments, the display and alarm module comprises a touch display. According to some embodiments, the display and alarm module is arranged and configured to provide an audible and visual alarm.

The explosion-proof enclosure may further comprise a power indicator for displaying a current power level of the air quantity analyzer for use in underground coal mines. In some embodiments, the explosion-proof enclosure further comprises a switch for turning the air quantity analyzer for use in underground coal mines off and on. According to some embodiments, the explosion-proof enclosure further comprises a wired transmission module interface for interfacing with the wired transmission module.

The power conversion module may further comprise at least one rechargeable lithium battery arranged and configured to supply power to an electrical component selected from the group consisting of the light source, the vacuum pump, the display and alarm module, the data communication module, an airflow velocity sensor, a laser profiler, and combinations thereof.

In some embodiments, the air quantity analyzer for use in underground coal mines further comprises a second air quantity analyzer for use in underground coal mines. According to some embodiments, the data communication module comprises a wired transmission module coupled to the second air quantity analyzer for use in underground coal mines. The data communication module may comprise a wireless transmission module coupled to the second air quantity analyzer for use in underground coal mines.

In some embodiments, the display and alarm module comprises a touch display. According to some embodiments, the display and alarm module is arranged and configured to provide an audible and visual alarm.

The explosion-proof enclosure may further comprise a power indicator for displaying a current power level of the air quantity analyzer for use in underground coal mines. In some embodiments, the explosion-proof enclosure further comprises a switch for turning the air quantity analyzer for use in underground coal mines off and on. According to some embodiments, the explosion-proof enclosure further comprises a wired transmission module interface for interfacing with the wired transmission module.

The power conversion module may further comprise at least one rechargeable lithium battery arranged and configured to supply power to an electrical component selected from the group consisting of the light source, the vacuum pump, the display and alarm module, the data communication module, an airflow velocity sensor, a laser profiler, and combinations thereof.

The disclosure also includes an air quantity analysis method for use in underground coal mines, comprising determining a rate of continuous release of tracer gas in cubic meters per minute. In some embodiments, the air quantity analysis method for use in underground coal mines comprises determining a distance selected from the group consisting of a distance between a tracer gas release point, a sampling point, and an air leakage point, a distance between the release point and the sampling point, in meters, and combinations thereof. According to some embodiments, the air quantity analysis method for use in underground coal mines comprises performing a test run of at least one air quantity analyzer. The air quantity analysis method for use in underground coal mines may comprise ensuring, via performing the test run, that an air sampler is not blocked and a vacuum pump operates normally. In some embodiments, the air quantity analysis method for use in underground coal mines comprises charging the at least one air quantity analyzer fully. According to some embodiments, the air quantity analysis method for use in underground coal mines comprises running the at least one air quantity analyzer at a selected placing point. The air quantity analysis method for use in underground coal mines may comprise turning on a laser profiler and an airflow velocity sensor. In some embodiments, the air quantity analysis method for use in underground coal mines comprises obtaining, via the laser profiler and the velocity sensor, an airflow velocity, a roadway cross-sectional area, and a shaft perimeter. According to some embodiments, the air quantity analysis method for use in underground coal mines comprises displaying, via a touch display, the rate of continuous release of tracer gas and the distance selected from the group consisting of the distance between a tracer gas release point, the sampling point, and the air leakage point, the distance between the release point and the sampling point, and combinations thereof. The air quantity analysis method for use in underground coal mines may comprise inputting, via the touch display, an interval time of a concentration sampling of tracer gas and a number of sampling times.

In some embodiments, the air quantity analysis method for use in underground coal mines further comprises detecting a local positive-pressure air leakage. According to some embodiments, the air quantity analysis method for use in underground coal mines further comprises determining a first continuous quantitative release point of tracer gas and a first release volume by determining the rate of continuous release of tracer gas and the distance selected from the group consisting of the distance between a tracer gas release point, the sampling point, and the air leakage point, the distance between the release point and the sampling point, and combinations thereof. The air quantity analysis method for use in underground coal mines may further comprise releasing, via a releasing device, a first predetermined quantity of tracer gas. In some embodiments, the air quantity analysis method for use in underground coal mines further comprises detecting, via a first air quantity analyzer, a relationship between a first air quantity and a first number of sampling times. According to some embodiments, the air quantity analysis method for use in underground coal mines further comprises recording, via the first air quantity analyzer, a curve of the relationship between the first air quantity and the first number of sampling times. The air quantity analysis method for use in underground coal mines may further comprise determining, via the first air quantity analyzer, if a first tracer gas concentration has exceeded a threshold tracer gas concentration. In some embodiments, the air quantity analysis method for use in underground coal mines further comprises sounding a first alarm in response to determining that the first tracer gas concentration has exceeded the threshold tracer gas concentration. According to some embodiments, the air quantity analysis method for use in underground coal mines further comprises shutting down the first air quantity analyzer in response to determining that the first tracer gas concentration has exceeded the threshold tracer gas concentration.

The air quantity analysis method for use in underground coal mines may further comprise determining a second continuous quantitative release point of tracer gas and a second release volume by determining the rate of continuous release of tracer gas and the distance selected from the group consisting of the distance between a tracer gas release point, the sampling point, and the air leakage point, the distance between the release point and the sampling point, and combinations thereof. In some embodiments, the air quantity analysis method for use in underground coal mines further comprises releasing, via the releasing device, a second predetermined quantity of tracer gas. According to some embodiments, the air quantity analysis method for use in underground coal mines further comprises detecting, via a second air quantity analyzer, a relationship between a second air quantity and a second number of sampling times. The air quantity analysis method for use in underground coal mines may further comprise recording, via the second air quantity analyzer, the curve of the relationship between the second air quantity and the second number of sampling times. In some embodiments, the air quantity analysis method for use in underground coal mines further comprises determining, via the second air quantity analyzer, if a second tracer gas concentration has exceeded the threshold tracer gas concentration. According to some embodiments, the air quantity analysis method for use in underground coal mines further comprises sounding a second alarm in response to determining that the second tracer gas concentration has exceeded the threshold tracer gas concentration. The air quantity analysis method for use in underground coal mines may further comprise shutting down the second air quantity analyzer in response to determining that the second tracer gas concentration has exceeded the threshold tracer gas concentration.

In some embodiments, the air quantity analysis method for use in underground coal mines further comprises displaying, via the touch display, an air leakage quantity under positive-pressure leakage in cubic meters per minute and an air leakage rate as a percentage. According to some embodiments, the air quantity analysis method for use in underground coal mines further comprises outputting, via the touch display, a curve of a relationship between the number of sampling times and the air leakage volume. The air quantity analysis method for use in underground coal mines may further comprise outputting, via the touch display, a curve of a relationship between the number of sampling times and the air quantity. In some embodiments, the air quantity analysis method for use in underground coal mines further comprises displaying, via the touch display, a table of air leakage detection results.

According to some embodiments, the air quantity analysis method for use in underground coal mines further comprises detecting a local negative-pressure air leakage. The air quantity analysis method for use in underground coal mines may further comprise determining a first continuous quantitative release point of tracer gas and a first release volume by determining the rate of continuous release of tracer gas and the distance selected from the group consisting of the distance between a tracer gas release point, the sampling point, and the air leakage point, the distance between the release point and the sampling point, and combinations thereof. In some embodiments, the air quantity analysis method for use in underground coal mines further comprises releasing, via a releasing device, a first predetermined quantity of tracer gas. According to some embodiments, the air quantity analysis method for use in underground coal mines further comprises detecting, via a first air quantity analyzer, a relationship between a first air quantity and a first number of sampling times. The air quantity analysis method for use in underground coal mines may further comprise recording, via the first air quantity analyzer, a curve of the relationship between the first air quantity and the first number of sampling times. In some embodiments, the air quantity analysis method for use in underground coal mines further comprises determining, via the first air quantity analyzer, if a first tracer gas concentration has exceeded a threshold tracer gas concentration. According to some embodiments, the air quantity analysis method for use in underground coal mines further comprises sounding a first alarm in response to determining that the first tracer gas concentration has exceeded the threshold tracer gas concentration. The air quantity analysis method for use in underground coal mines may further comprise recording, via the first air quantity analyzer, the first tracer gas concentration in response to determining that the first tracer gas concentration has exceeded the threshold tracer gas concentration.

In some embodiments, the air quantity analysis method for use in underground coal mines further comprises determining a second continuous quantitative release point of tracer gas and a second release volume by determining the rate of continuous release of tracer gas and the distance selected from the group consisting of the distance between a tracer gas release point, the sampling point, and the air leakage point, the distance between the release point and the sampling point, and combinations thereof. According to some embodiments, the air quantity analysis method for use in underground coal mines further comprises releasing, via the releasing device, a second predetermined quantity of tracer gas. The air quantity analysis method for use in underground coal mines may further comprise detecting, via a second air quantity analyzer, a relationship between a second air quantity and a second number of sampling times. In some embodiments, the air quantity analysis method for use in underground coal mines further comprises recording, via the second air quantity analyzer, the curve of the relationship between the second air quantity and the second number of sampling times. According to some embodiments, the air quantity analysis method for use in underground coal mines further comprises determining, via the second air quantity analyzer, if a second tracer gas concentration has exceeded the threshold tracer gas concentration. The air quantity analysis method for use in underground coal mines may further comprise sounding a second alarm in response to determining that the second tracer gas concentration has exceeded the threshold tracer gas concentration. In some embodiments, the air quantity analysis method for use in underground coal mines further comprises recording, via the second air quantity analyzer, the second tracer gas concentration in response to determining that the second tracer gas concentration has exceeded the threshold tracer gas concentration. According to some embodiments, the air quantity analysis method for use in underground coal mines further comprises stopping detection.

The air quantity analysis method for use in underground coal mines may further comprise connecting, via the transmission module selected from the group consisting of the wired transmission module and the wireless transmission module, the first air quantity analyzer, the second air quantity analyzer, and combinations thereof. In some embodiments, the air quantity analysis method for use in underground coal mines further comprises facilitating information transfer by connecting the first air quantity analyzer and the second air quantity analyzer.

According to some embodiments, the air quantity analysis method for use in underground coal mines further comprises displaying, via the touch display, an air leakage quantity under negative-pressure leakage in cubic meters per minute and an air leakage rate as a percentage. The air quantity analysis method for use in underground coal mines may further comprise outputting, via the touch display, a curve of a relationship between the number of sampling times and the air leakage volume. In some embodiments, the air quantity analysis method for use in underground coal mines further comprises outputting, via the touch display, a curve of a relationship between the number of sampling times and the air quantity. According to some embodiments, the air quantity analysis method for use in underground coal mines further comprises displaying, via a touch display, a table of air leakage detection results.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a schematic diagram of the appearance and structure of the air quantity analyzer in underground coal mines, according to some embodiments.

FIG. 2 illustrates a schematic diagram of the working principle of the air quantity analyzer in underground coal mines, according to some embodiments.

FIG. 3 illustrates a schematic diagram of the circuit for the audible and visual alarm, according to some embodiments.

FIG. 4 illustrates a schematic diagram of local positive-pressure air leakage detection, according to some embodiments.

FIG. 5 illustrates a schematic diagram of local negative-pressure air leakage detection, according to some embodiments.

FIGS. 6 and 7 illustrate graphs showing the relationship between the number of sampling times and the leakage quantity, according to some embodiments.

FIG. 8 illustrates a graph showing the relationship between the number of sampling times and the sulfur hexafluoride (SF₆) concentration under negative-pressure air leakage, according to some embodiments.

FIG. 9 illustrates a graph showing the relationship between the number of sampling times and the sulfur hexafluoride (SF₆) concentration under positive-pressure air leakage, according to some embodiments.

FIG. 10 illustrates a flowchart depicting a method of setting up an air quantity analyzer, according to some embodiments.

FIG. 11 illustrates a flowchart depicting a method of analyzing a first air quantity under local positive-pressure air leakage, according to some embodiments.

FIG. 12 illustrates a flowchart depicting a method of analyzing a second air quantity under local positive-pressure air leakage, according to some embodiments.

FIG. 13 illustrates a flowchart depicting a method of displaying information about local positive-pressure air leakage, according to some embodiments.

FIG. 14 illustrates a flowchart depicting a method of analyzing a first air quantity under local negative-pressure air leakage, according to some embodiments.

FIG. 15 illustrates a flowchart depicting a method of analyzing a second air quantity under local negative-pressure air leakage, according to some embodiments.

FIG. 16 illustrates a flowchart depicting a method of information transfer between air quantity analyzers and displaying information about local positive-pressure air leakage, according to some embodiments.

### DETAILED DESCRIPTION

Although specific embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order-dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### COMPONENT INDEX

1—Explosion-proof enclosure
2—Air sampler
3—Sampling head
4—Current regulator
5—Dust and moisture removal device
8—Spiral gas circuit
9—Power indicator
10—Switch
12—Airflow velocity sensor
13—Laser profiler
16—Central processor
20—Power conversion module
30—Detection device
31—Light source
32—Gas pool
33—Optical path
34—Detector
35—Vacuum pump
39—Gas evacuation end
40—Data communication module
41—Wireless transmission module
42—Wired transmission module
50—Touch display
51—Display and alarm module
52—Sound and light alarm

Air leakage in coal mines may induce air quantity fluctuations, deterioration of climatic and sanitary conditions at the working face or air-use sites, and even additional electrical energy consumption. However, a more significant issue is that air leakage accelerates the oxidation of float coal in the goaf, which is a primary cause of most mine fires.

Currently, coal mines in China mainly use the tracer technique to detect leakage channels and measure the leakage quantity. This tracer technique is generally practiced as follows: first, one selects sulfur hexafluoride (SF₆) as the tracer gas and considers the airflow or leaking air as the carrier. Next, manually release the SF₆ through a flow meter at a leaking source that has a relatively high energy level. Third, collect gas samples from possible outlets for analysis to ascertain the tracer gas trajectory. Next, identify the leakage channel. Finally, calculate the leakage quantity based on the change in concentration of the tracer gas.

However, in practice, there is no specialized air leakage detecting device for use in coal mines, so the identification of leakage channels and the amount of leakage can only be performed by a human who takes the gas samples on-site and then sends them to a laboratory for analysis. The results of this analysis may fail to reflect the actual air quantity faithfully and leakage in the underground coal mines as a result of one or more interference factors, including but not limited to: uncertainty in the sampling time point, the sampling time can only be estimated by sampling operators who may miss some sampling points and leakage channels or take incorrect gas samples due to human error which would affect the testing results of the air leakage; that the SF₆ gas in the sampling bag may be affected by adsorption, leakage, and gas mixing; that the chromatograph with an electronic trap that is used for quantitative analysis of SF₆ gas requires periodic calibration and as such may be prone to error depending on last service date; and that the analysis results may easily be distorted by an operator(s). Additionally, the operators may need to occupy space in the roadway that could be otherwise utilized.

The specific analysis method is described in China's coal industry-standard MT/T845-1999 Technical Specification of Leakage Detection with SF₆ Tracer Gas in Roadways of Coal Mines: determine the q value of continuous quantitative release of SF₆ tracer gas in cubic meters per minute (m³/min), and obtain the L value of the distance between the tracer gas release point and the sampling point and the air leakage point, or between the release point and the sampling point, in meters (m).

Determine the q value:

q=KCQ
where q is the continuous quantitative release of SF₆ tracer gas in m³/min;
K is the coefficient whose value ranges from 4 to 5;
C is the minimum SF₆ concentration in the predetermined air quantity whose value ranges from 8 to 10; and
Q is the air quantity through the tested roadways m³/min.

Q=V·S
where V is the airflow velocity in m/min; and
S is the roadway cross-sectional area in m².

Calculate the L value:

$$L \geq \frac{32 S}{U}$$

where L is the distance between the tracer gas release point and the sampling point and the air leakage point, or between the release point and the sampling point in m;
S is the roadway cross-sectional area in m²; and
U is the shaft perimeter in m.

Calculating the ΔQ value under local positive-pressure leakage or local negative-pressure leakage:

$$Δ Q = \frac{q ( {c}_{2} - {c}_{1} )}{{c}_{1} - {c}_{2}}$$

where ΔQ is the air leakage quantity in the R1-R2 section of the detected roadway under positive-pressure leakage in m³/min, or the air leakage quantity in the S1-S2 section of the detected roadway under negative-pressure leakage in m³/min;
q is the released quantity of the tracer gas SF₆ in m³/min;
c₁ is the concentration of SF₆ at measuring point 1; and
c₂ is the concentration of SF₆ at measuring point 2.

Calculating the α value:

$$α = \frac{{c}_{2} - {c}_{1}}{{c}_{2}} \cdot 100 \%$$

where α is the air leakage rate of the detected roadway in percent (%);
c₁ is the concentration of SF₆ at measuring point 1; and
c₂ is the concentration of SF₆ at measuring point 2, wherein
a positive α value indicates positive-pressure leakage, and
a negative α value indicates negative-pressure leakage.

This invention will not elaborate on the calculation of ΔQᵢ or αᵢ under continuous positive-pressure leakage or continuous negative-pressure leakage. For more details, please refer to pages 3, 4, and 6 of “Handling of Detection Results” in China's coal industry standard MT/T845-1999 Technical Specification of Leakage Detection with SF₆ Tracer Gas in Roadways of Coal Mines.

The rationale of Fourier Transform Infrared Spectroscopy (FTIR) SF₆ detection is as follows: SF₆, as a polar gas, has strong absorption characteristics for infrared light in specific wavelengths. FTIR is characterized by high sensitivity, continuity of detection, immunization from environmental influence and interference, and minor detection errors caused by changes in ambient temperature and humidity. At present, the application of FTIR to the detection of SF₆ concentration in underground coal mines is not yet seen throughout the world.

One purpose of the invention described herein is to provide a portable, intrinsically safe, and explosion-proof air quantity analyzer and analysis method used in underground coal mines to find air leakage channels and perform quantitative analysis on air leakage. The analyzer and its method of analysis function through the use of spectral analysis technology to achieve rapid quantitative detection of air quantity, roadway cross-sectional area, and air leakage, as well as automatic detection and alarming, continuous monitoring, underground in-situ testing, on-site results analysis, and automatic data transmission, among other possible use cases. These facets make the analyzer and analysis method especially suitable for underground coal mines with complex ventilation.

According to some embodiments, in this air quantity analyzer, the gas pipeline connects the air sampler, the gas pool, the vacuum pump of the detection device in the explosion-proof enclosure, and the gas evacuation end. The central processor in the explosion-proof enclosure may be electrically coupled to the light source, the detector, the vacuum pump of the detection device, the power conversion module, the display and alarm module, and the data communication module. One of the novel facets of this invention is that the air sampler may comprise a sampling head, a current regulator, a dust and moisture removal device, and a spiral gas circuit, coupled to one another in the order as listed.

In some embodiments, the central processor is electrically coupled to the laser profiler and the airflow velocity sensor. The data communication module may include a wired transmission module and a wireless transmission module, both of which may be coupled to another underground coal mine air quantity analyzer. According to some embodiments, the display and alarm module includes a touch display and an audible and visible alarm. In some embodiments, the explosion-proof enclosure has a power indicator, a switch, and a wired transmission module interface. The power conversion module may include rechargeable lithium batteries that supply power to the light source, the vacuum pump, the display and alarm module, the data communication module, the airflow velocity sensor, and the laser profiler.

The invention also includes a method of analysis for the air quantity analyzer in underground coal mines. In some embodiments, the method comprises includes determining the q value for the continuous quantitative release of SF₆ tracer gas in m³/min, according to China's coal industry standard MT/T845-1999 Technical Specification of Leakage Detection with SF₆ Tracer Gas in Roadways of Coal Mines, as well as determining the L value of the distance between the tracer gas release point and the sampling point and the air leakage point, or between the release point and the sampling point in m. According to some embodiments, first, test runs of the air quantity analyzer are performed to make sure the air sampler is not blocked and the vacuum pump operates normally. After being fully charged, the air quantity analyzer may run at a selected pacing point. In some embodiments, the laser profiler and the airflow velocity sensor are turned on next to obtain the airflow velocity V, the roadway cross-sectional area S, and the shaft perimeter U. The q and L values may then be shown on the touch display. The interval time Δt of the concentration sampling of tracer gas SF₆ and the number of sampling times n may be input into the touch display.

According to some embodiments, in detecting local positive-pressure air leakage, first, the continuous quantitative release point R1 of SF₆ tracer gas and the release volume are determined according to the q value and the L value. In some embodiments, as the releasing device releases SF₆ tracer gas in the predetermined quantity, the air quantity analyzer begins detecting and recording the curve of the relationship between Qⱼ of air quantity Q and the number of sampling times n (or time t). When the air quantity analyzer detects SF₆ tracer gas in a concentration greater than one trillion (C1>10¹⁰), it may sound an alarm and record the C1ⱼ value from measuring point 1 to form a C1ⱼ relationship curve between sampling times n and C1 of SF₆ tracer gas concentration, and then it may shut down. In some embodiments, the next step is to move the continuous quantitative release point to R2 and repeat these steps. According to some embodiments, the analyzer stops detecting when it has drawn the C2ⱼ curve of the relationship between the number of sampling times n and C2 of SF₆ tracer gas concentration.

The C1 and C2 values may be automatically calculated according to the following principles and equations:

$$\overline{C} 1 = \frac{{\sum }_{j = N}^{N + {n}_{0} - 1} C {1}_{j}}{N + {n}_{0} - 1} , ( N = 1 , 2 , … {n}_{1} , … {n}_{2} , … n + {n}_{0} - 1 ) \\ \overline{C} 2 = \frac{{\sum }_{j = N}^{N + {n}_{0} - 1} C {2}_{j}}{N + {n}_{0} - 1} , ( N = 1 , 2 , … {n}_{1} , … {n}_{2} , … n + {n}_{0} - 1 ) \\ while \\ \frac{\vert \overline{C} 1 - C {1}_{j} \vert}{\overline{C}} \leq 5 \% , ( j = N … N + {n}_{0} - 1 ) , and \\ \frac{\vert \overline{C} 2 - C {2}_{j} \vert}{\overline{C}} \leq 5 \% , ( j = N … N + {n}_{0} - 1 ) \\ C 1 = \overline{C} 1 , ( {n}_{1} = N , {n}_{2} = N + {n}_{0} - 1 ) \\ C 2 = \overline{C} 2 , ( {n}_{1} = N , {n}_{2} = N + {n}_{0} - 1 )$$

Wherein no represents the number of sampling times at each detection point, time, and input from the touch display. The touch display may show the ΔQ and α values and output the curve of relationships between the number of sampling times and the air leakage volume, and the curve of relationships between the number of sampling times (or time) and air quantity, as well as the table of air leakage detection results.

According to some embodiments, in detecting local negative-pressure air leakage, first, the continuous quantitative release point R of SF₆ tracer gas and the release volume are determined according to the q value and the L value. In some embodiments, as the releasing device releases SF₆ tracer gas in the predetermined quantity, air quantity analyzers at S1 and S2 begin detection. In some embodiments, the air quantity analyzer at S2 begins detecting and recording the curve of the relationship between Q; of air quantity Q and the number of sampling times n (or time t). When the air quantity analyzer at S2 detects SF₆ tracer gas in a concentration greater than one trillion (C2>10¹⁰), it may sound an alarm and record the C2ⱼ value from measuring point 1 to form a C2ⱼ relationship curve between sampling times n and C2 of SF₆ tracer gas concentration. According to some embodiments, when the air quantity analyzer at S1 detects SF₆ tracer gas in a concentration greater than one trillion (C1>101′), it will sound an alarm and record the C1ⱼ value from measuring point 1 to form a C1ⱼ relationship curve between sampling times n and C1 of SF₆ tracer gas concentration. In some embodiments, the detections at C1 and C2 co-occur. After these detections are performed, the detection may stop. According to some embodiments, the analyzers can be connected with each other through either a wired transmission module or a wireless transmission module, either of which can facilitate information transfer between analyzers.

Once again, the C1 and C2 values may be automatically calculated according to the following principles and equations:

$$\overline{C} 1 = \frac{{\sum }_{j = N}^{N + {n}_{0} - 1} C {1}_{j}}{N + {n}_{0} - 1} , ( N = 1 , 2 , … {n}_{1} , … {n}_{2} , … n + {n}_{0} - 1 ) \\ \overline{C} 2 = \frac{{\sum }_{j = N}^{N + {n}_{0} - 1} C {2}_{j}}{N + {n}_{0} - 1} , ( N = 1 , 2 , … {n}_{1} , … {n}_{2} , … n + {n}_{0} - 1 ) \\ while \\ \frac{\vert \overline{C} 1 - C {1}_{j} \vert}{\overline{C}} \leq 5 \% , ( j = N … N + {n}_{0} - 1 ) , and \\ \frac{\vert \overline{C} 2 - C {2}_{j} \vert}{\overline{C}} \leq 5 \% , ( j = N … N + {n}_{0} - 1 ) \\ C 1 = \overline{C} 1 , ( {n}_{1} = N , {n}_{2} = N + {n}_{0} - 1 ) \\ C 2 = \overline{C} 2 , ( {n}_{1} = N , {n}_{2} = N + {n}_{0} - 1 )$$

Wherein n₀ represents the number of sampling times at each detection point, time, and input from the touch display. The touch display may show the ΔQ and α values and output the curve of relationships between the number of sampling times and the air leakage volume, and the curve of relationships between the number of sampling times (or time) and air quantity, as well as the table of air leakage detection results.

Points of novelty, as well as benefits of the herein described air quantity analyzer for underground coal mines and the method of analysis, include high detection accuracy, a large data storage volume, automatic sampling, continuous detection, simplistic operation, automatic reporting, and accurate detection of the air quantity and analysis of the air leakage status. Additionally, the invention may be used in underground coal mines for on-site detection and analysis without being affected by large underground mechanical or electrical equipment.

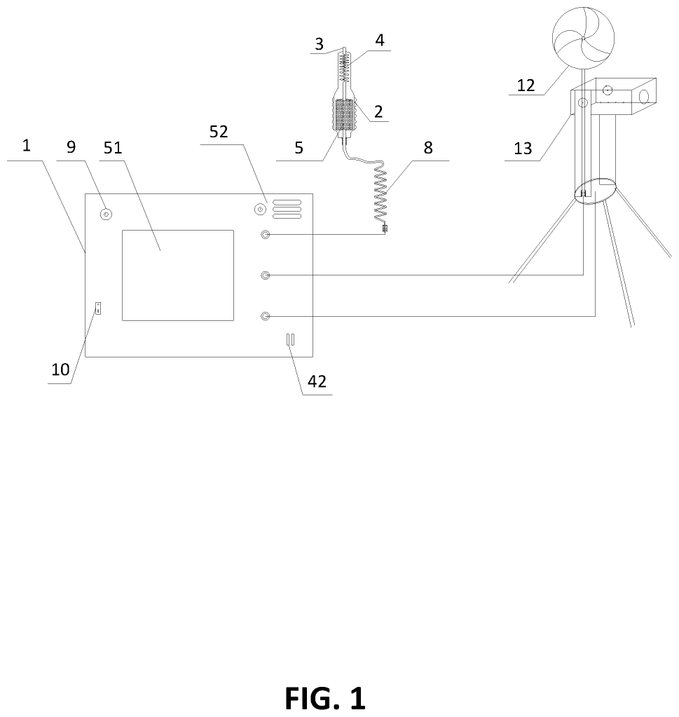

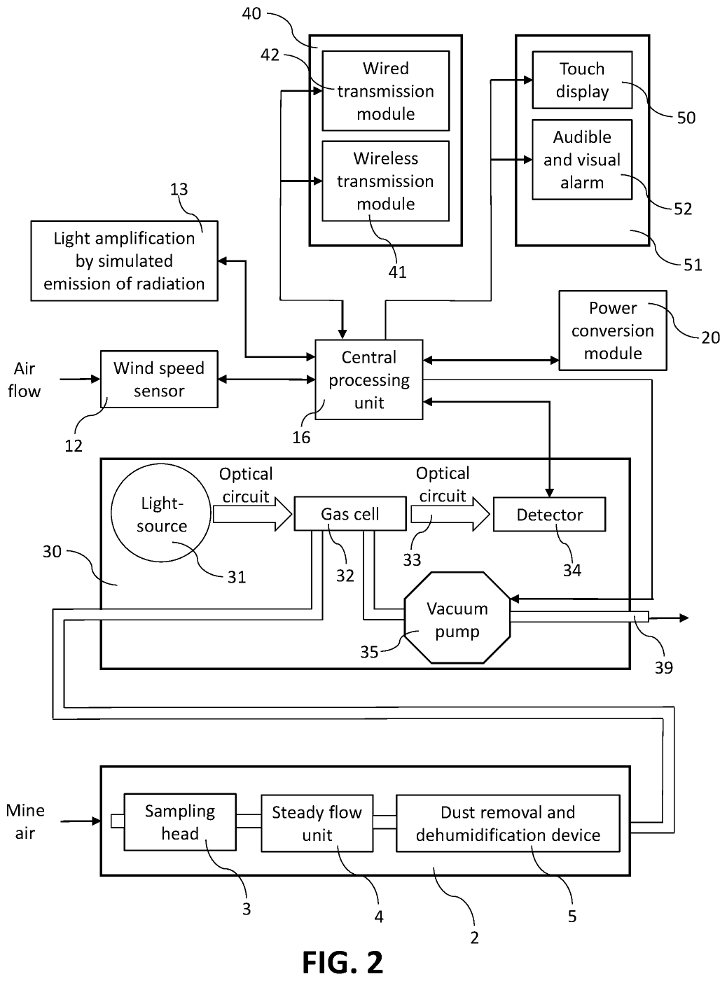

As illustrated in FIGS. 1-3, a gas pipeline may connect the air sampler 2, the gas pool 32, the vacuum pump 35 of the detection device 30 in the explosion-proof enclosure 1. And the gas evacuation end 39. According to some embodiments, the central processor 16 inside the explosion-proof enclosure 1 is ARM2440 type and is electrically coupled to the light source 31, the detector 34, and the vacuum pump 35 of the detection device 30. In some embodiments, the power conversion module 20 includes rechargeable lithium batteries, the display and alarm module 51, and the data communication module 40. The air sampler 2 may comprise a sampling head 3, a current regulator 4, a dust and moisture removal device 5, and a spiral gas circuit 8, coupled to one another in the order as listed. In some embodiments, detector 34 detects the spectrum of the light path 33 emitted by the light source 31 through the gas pool 32.

According to some embodiments, the ARM2440 central processor 16 is also electrically coupled to a DXC laser profiler 13 and a GFW15 airflow velocity sensor 12.

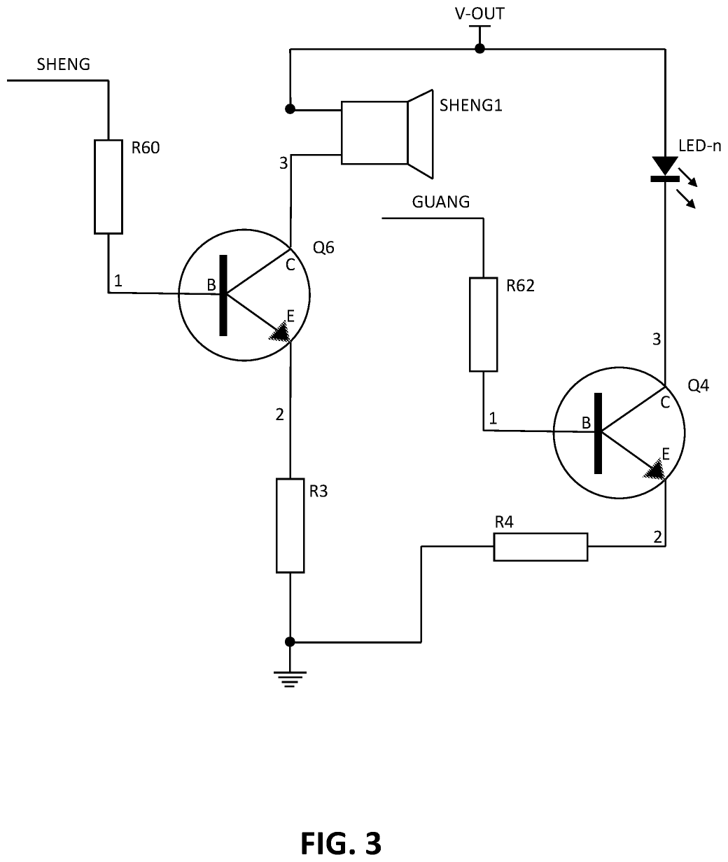

The data communication module 40 may include a wired transmission module 42 and a wireless transmission module 41, both of which may be connected to another air quantity analyzer, thus achieving information transfer between two or more air quantity analyzers. In some embodiments, the display and alarm module 51 includes a touch display 50 and an audible and visual alarm 52. FIG. 3 illustrates a general circuit for these components. According to some embodiments, the surface of the explosion-proof enclosure 1 includes a power indicator 9, a switch 10, and a wired transmission module 42 interface.

The power conversion module 20 may include rechargeable lithium batteries to supply power to light source 31, vacuum pump 35, display alarm module 51, the data communication module 40, GFW15 airflow velocity sensor 12, and DXC laser profiler 13.

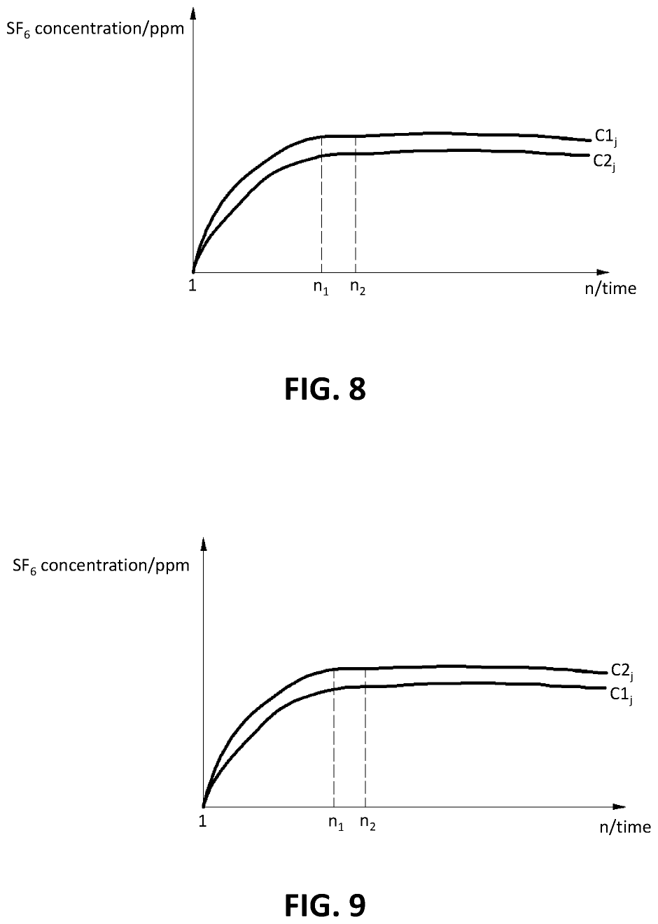

The analysis method as illustrated in FIGS. 1-9 is described in China's coal industry standard MT/T845-1999 Technical Specification of Leakage Detection with SF₆ Tracer Gas in Roadways of Coal Mines, reiterated here for convenience: determine the q value of continuous quantitative release of SF₆ tracer gas in cubic meters per minute (m³/min), and obtain the L value of the distance between the tracer gas release point and the sampling point and the air leakage point, or between the release point and the sampling point, in meters (m).

Determine the q value:

q=KCQ
where q is the continuous quantitative release of SF₆ tracer gas in m³/min;
K is the coefficient whose value ranges from 4 to 5;
C is the minimum SF₆ concentration in the predetermined air quantity whose value ranges from 8 to 10; and
Q is the air quantity through the tested roadways in m³/min.

Q=V·S
where V is the airflow velocity in m/min; and
S is the roadway cross-sectional area in m².

Calculate the L value:

$$L \geq \frac{32 S}{U}$$

where L is the distance between the tracer gas release point and the sampling point and the air leakage point, or between the release point and the sampling point in m;
S is the roadway cross-sectional area in m²; and
U is the shaft perimeter in m.

Calculating the ΔQ value under local positive-pressure leakage or local negative-pressure leakage:

$$Δ Q = \frac{q ( {c}_{2} - {c}_{1} )}{{c}_{1} - {c}_{2}}$$

where ΔQ is the air leakage quantity in the R1-R2 section of the detected roadway under positive-pressure leakage in m³/min, or the air leakage quantity in the S1-S2 section of the detected roadway under negative-pressure leakage in m³/min;
q is the released quantity of the tracer gas SF₆ in m³/min
c₁ is the concentration of SF₆ at measuring point 1; and
c₂ is the concentration of SF₆ at measuring point 2.

Calculating the α value:

$$α = \frac{{c}_{2} - {c}_{1}}{{c}_{2}} \cdot 100 \%$$

where α is the air leakage rate of the detected roadway in percent (%);
c₁ is the concentration of SF₆ at measuring point 1; and
c₂ is the concentration of SF₆ at measuring point 2, wherein
a positive α value indicates positive-pressure leakage, and
a negative α value indicates negative-pressure leakage.

These calculations may be performed by the central processor 16 after programming in these formulae. According to some embodiments, the method includes performing test runs on the air quantity analyzer to ensure that the air sampler 2 is not blocked and the vacuum pump 35 operates normally. In some embodiments, after being fully charged, the air quantity analyzer runs at a selected placing point. Then, the laser profiler 13 and the airflow velocity sensor 12 may be turned on so as to obtain the airflow velocity V, the roadway cross-sectional area S, and the shaft perimeter U. In some embodiments, the q and L values are shown on the touch display 50, and the interval time Δt of the concentration sampling of tracer gas SF₆ and the number of sampling times n are input into the touch display 50.

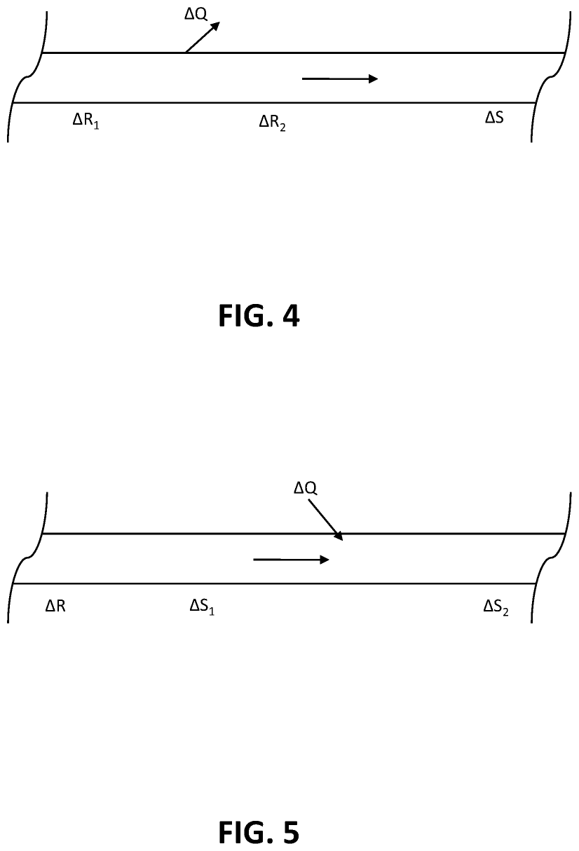

FIGS. 4, 5, 6, and 9 illustrate local positive-pressure air leakage detection, according to some embodiments. First, the continuous quantitative release point R1 of SF₆ tracer gas and the release volume may be determined according to the q and L values. In some embodiments, as the releasing device releases SF₆ tracer gas in the predetermined quantity, the air quantity analyzer begins detecting and recording the curve of the relationship between Qⱼ of air quantity Q and the number of sampling times n (or time t). When the air quantity analyzer detects SF₆ tracer gas in a concentration greater than one trillion (C1>10¹⁰), it may sound an alarm and record the C1ⱼ value from measuring point 1 to form a C1ⱼ relationship curve between sampling times n and C1 of SF₆ tracer gas concentration, and then it may shut down. In some embodiments, the next step is to move the continuous quantitative release point to R2 and repeat these steps. According to some embodiments, the analyzer stops detecting when it has drawn the C2ⱼ curve of the relationship between the number of sampling times n and C2 of SF₆ tracer gas concentration. The formulas will be provided for convenience on the next page.

FIGS. 5-8 illustrate detection of local negative-pressure air leakage, according to some embodiments. First, the continuous quantitative release point R of SF₆ tracer gas and the release volume may be determined according to the q and L values. In some embodiments, as the releasing device releases SF₆ tracer gas in the predetermined quantity, air quantity analyzers at S1 and S2 begin detection. In some embodiments, the air quantity analyzer at S2 begins detecting and recording the curve of the relationship between Qₜ of air quantity Q and the number of sampling times n (or time t). When the air quantity analyzer at S2 detects SF₆ tracer gas in a concentration greater than one trillion (C2>10¹⁰), it may sound an alarm and record the C2ⱼ value from measuring point 1 to form a C2ⱼ relationship curve between sampling times n and C2 of SF₆ tracer gas concentration. According to some embodiments, when the air quantity analyzer at S1 detects SF₆ tracer gas in a concentration greater than one trillion (C1>10¹⁰), it will sound an alarm and record the C1ⱼ value from measuring point 1 to form a C1ⱼ relationship curve between sampling times n and C1 of SF₆ tracer gas concentration. In some embodiments, the detections at C1 and C2 occur simultaneously. After these detections are performed, the detection may stop. According to some embodiments, the analyzers can be connected with each other through either a wired transmission module or a wireless transmission module, either of which can facilitate information transfer between analyzers.

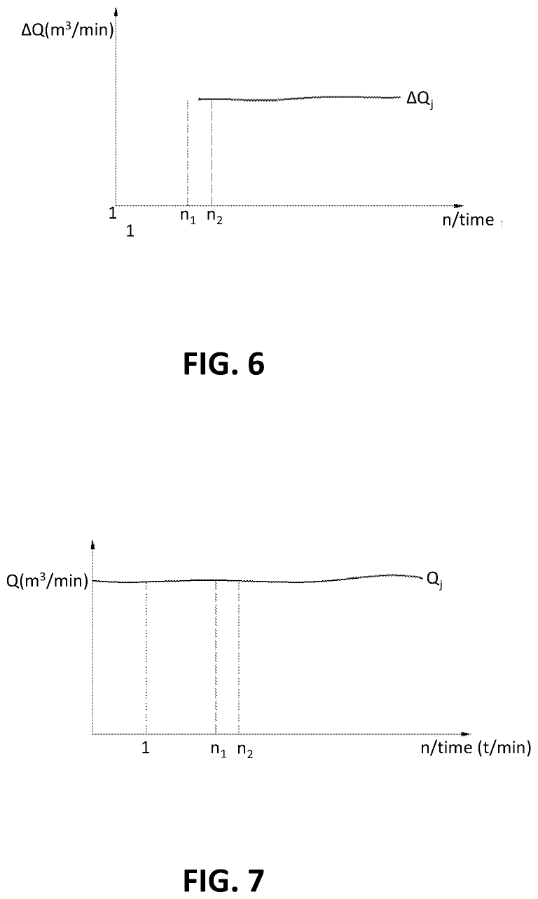

Provided here for convenience—the C1 and C2 values may be automatically calculated according to the following principles and equations:

$$\overline{C} 1 = \frac{{\sum }_{j = N}^{N + {n}_{0} - 1} C {1}_{j}}{N + {n}_{0} - 1} , ( N = 1 , 2 , … {n}_{1} , … {n}_{2} , … n + {n}_{0} - 1 ) \\ \overline{C} 2 = \frac{{\sum }_{j = N}^{N + {n}_{0} - 1} C {2}_{j}}{N + {n}_{0} - 1} , ( N = 1 , 2 , … {n}_{1} , … {n}_{2} , … n + {n}_{0} - 1 ) \\ while \\ \frac{\vert \overline{C} 1 - C {1}_{j} \vert}{\overline{C}} \leq 5 \% , ( j = N … N + {n}_{0} - 1 ) , and \\ \frac{\vert \overline{C} 2 - C {2}_{j} \vert}{\overline{C}} \leq 5 \% , ( j = N … N + {n}_{0} - 1 ) \\ C 1 = \overline{C} 1 , ( {n}_{1} = N , {n}_{2} = N + {n}_{0} - 1 ) \\ C 2 = \overline{C} 2 , ( {n}_{1} = N , {n}_{2} = N + {n}_{0} - 1 )$$

Wherein no represents the number of sampling times at each detection point, time, and input from the touch display 50. The touch display 50 may show the ΔQ and α values and output the curve of relationships between the number of sampling times and the air leakage volume (as illustrated in FIG. 6), and the curve of relationships between the number of sampling times (or time) and air quantity (as shown in FIG. 7), as well as the table of air leakage detection results.

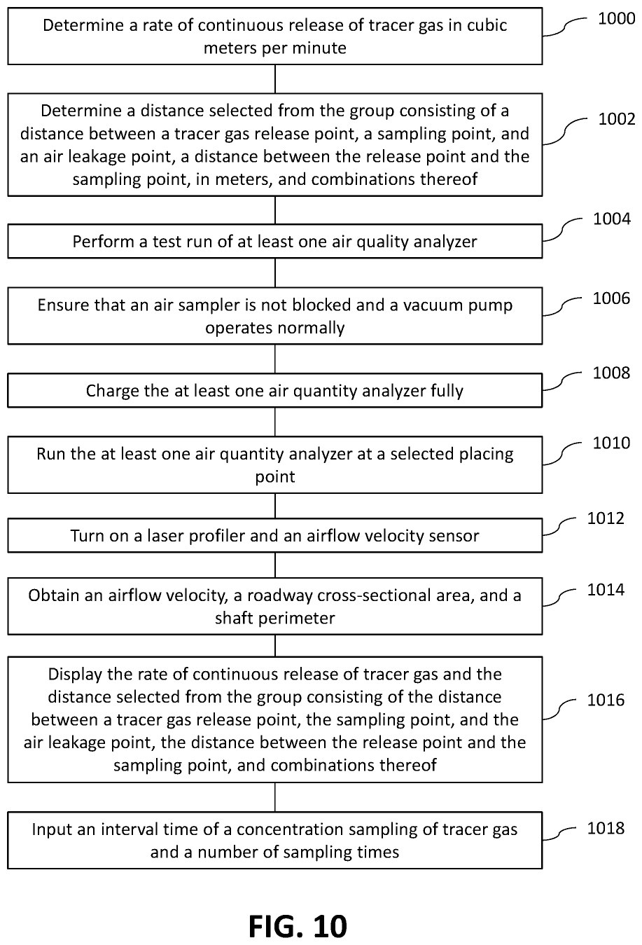

FIG. 10 illustrates a method of setting up an air quantity analyzer, according to some embodiments. In some embodiments, the method includes determining a rate of continuous release of tracer gas in cubic meters per minute (at step 1000). According to some embodiments, the method includes determining a distance selected from the group consisting of a distance between a tracer gas release point, a sampling point, and an air leakage point, a distance between the release point and the sampling point, in meters, and combinations thereof (at step 1002). The method may include performing a test run of at least one air quantity analyzer (at step 1004). In some embodiments, the method includes ensuring, via performing the test run, that an air sampler is not blocked and a vacuum pump operates normally (at step 1006). According to some embodiments, the method includes charging the at least one air quantity analyzer fully (at step 1008). The method may include running the at least one air quantity analyzer at a selected placing point (at step 1010). In some embodiments, the method includes turning on a laser profiler and an airflow velocity sensor (at step 1012). According to some embodiments, the method includes obtaining, via the laser profiler and the velocity sensor, an airflow velocity, a roadway cross-sectional area, and a shaft perimeter (at step 1014). The method may include displaying, via a touch display, the rate of continuous release of tracer gas and the distance selected from the group consisting of the distance between a tracer gas release point, the sampling point, and the air leakage point, the distance between the release point and the sampling point, and combinations thereof (at step 1016). In some embodiments, the method includes inputting, via the touch display, an interval time of a concentration sampling of tracer gas and a number of sampling times (at step 1018).

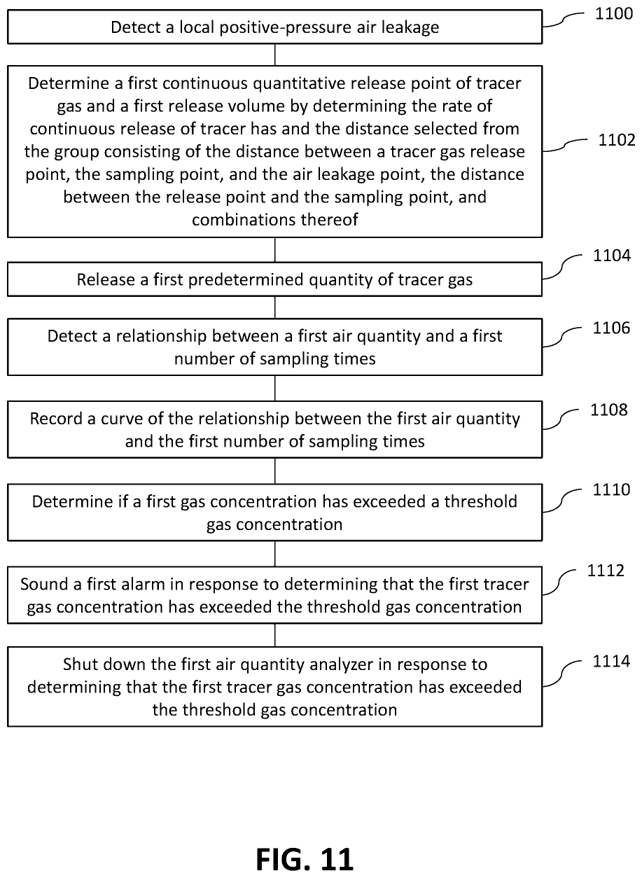

FIG. 11 illustrates a method of analyzing a first air quantity under local positive-pressure air leakage, according to some embodiments. According to some embodiments, the method includes detecting a local positive-pressure air leakage (at step 1100). The method may include determining a first continuous quantitative release point of tracer gas and a first release volume by determining the rate of continuous release of tracer gas and the distance selected from the group consisting of the distance between a tracer gas release point, the sampling point, and the air leakage point, the distance between the release point and the sampling point, and combinations thereof (at step 1102). In some embodiments, the method includes releasing, via a releasing device, a first predetermined quantity of tracer gas (at step 1104). According to some embodiments, the method includes detecting, via a first air quantity analyzer, a relationship between a first air quantity and a first number of sampling times (at step 1106). The method may include recording, via the first air quantity analyzer, a curve of the relationship between the first air quantity and the first number of sampling times (at step 1108). In some embodiments, the method includes determining, via the first air quantity analyzer, if a first tracer gas concentration has exceeded a threshold tracer gas concentration (at step 1110). According to some embodiments, the method includes sounding a first alarm in response to determining that the first tracer gas concentration has exceeded the threshold tracer gas concentration (at step 1112). The method may include shutting down the first air quantity analyzer in response to determining that the first tracer gas concentration has exceeded the threshold tracer gas concentration (at step 1114).

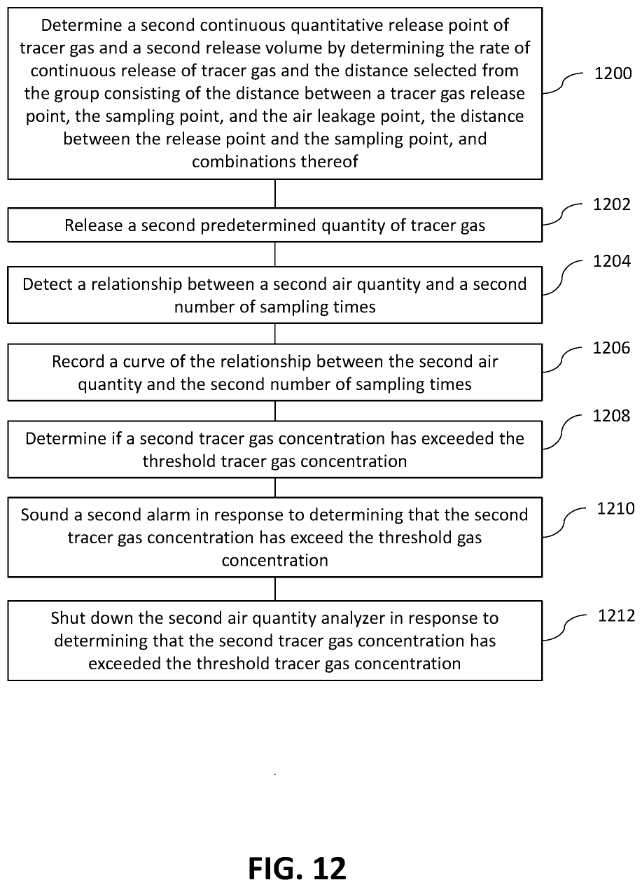

FIG. 12 illustrates a method of analyzing a second air quantity under local positive-pressure air leakage, according to some embodiments. In some embodiments, the method includes determining a second continuous quantitative release point of tracer gas and a second release volume by determining the rate of continuous release of tracer gas and the distance selected from the group consisting of the distance between a tracer gas release point, the sampling point, and the air leakage point, the distance between the release point and the sampling point, and combinations thereof (at step 1200). According to some embodiments, the method includes releasing, via the releasing device, a second predetermined quantity of tracer gas (at step 1202). The method may include detecting, via a second air quantity analyzer, a relationship between a second air quantity and a second number of sampling times (at step 1204). In some embodiments, the method includes recording, via the second air quantity analyzer, the curve of the relationship between the second air quantity and the second number of sampling times (at step 1206). According to some embodiments, the method includes determining, via the second air quantity analyzer, if a second tracer gas concentration has exceeded the threshold tracer gas concentration (at step 1208). The method may include sounding a second alarm in response to determining that the second tracer gas concentration has exceeded the threshold tracer gas concentration (at step 1210). In some embodiments, the method includes shutting down the second air quantity analyzer in response to determining that the second tracer gas concentration has exceeded the threshold tracer gas concentration (at step 1212).

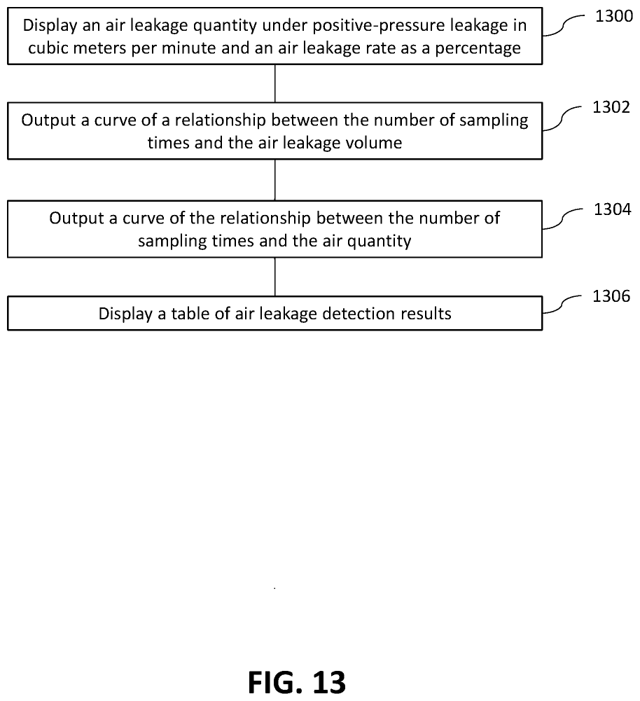

FIG. 13 illustrates a method of displaying information about local positive-pressure air leakage, according to some embodiments. According to some embodiments, the method includes displaying, via the touch display, an air leakage quantity under positive-pressure leakage in cubic meters per minute and an air leakage rate as a percentage (at step 1300). The method may include outputting, via the touch display, a curve of a relationship between the number of sampling times and the air leakage volume (at step 1302). In some embodiments, the method includes outputting, via the touch display, a curve of a relationship between the number of sampling times and the air quantity (at step 1304). According to some embodiments, the method includes displaying, via the touch display, a table of air leakage detection results (at step 1306).

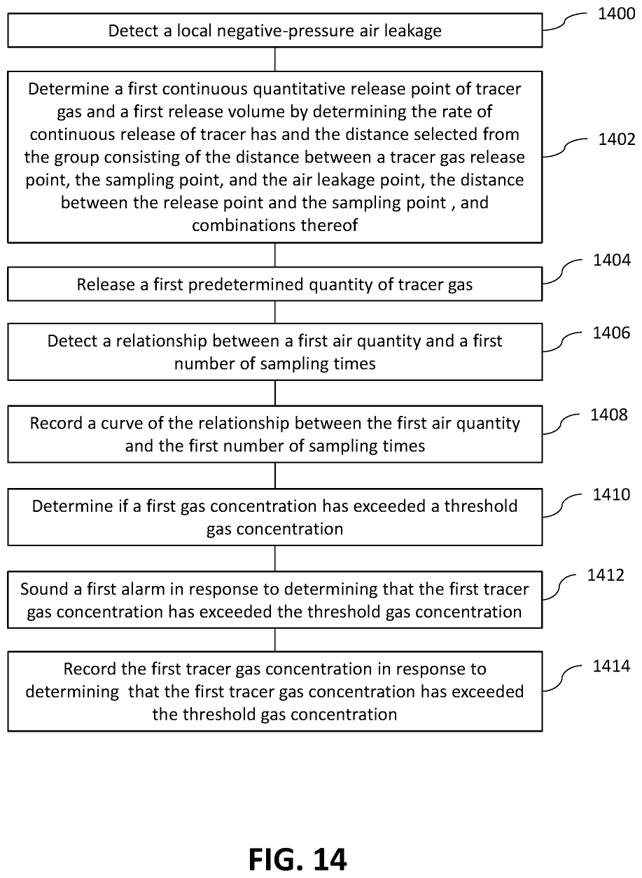

FIG. 14 illustrates a method of analyzing a first air quantity under local negative-pressure air leakage, according to some embodiments. The method may include detecting a local negative-pressure air leakage (at step 1400). In some embodiments, the method includes determining a first continuous quantitative release point of tracer gas and a first release volume by determining the rate of continuous release of tracer gas and the distance selected from the group consisting of the distance between a tracer gas release point, the sampling point, and the air leakage point, the distance between the release point and the sampling point, and combinations thereof (at step 1402). According to some embodiments, the method includes releasing, via a releasing device, a first predetermined quantity of tracer gas (at step 1404). The method may include detecting, via a first air quantity analyzer, a relationship between a first air quantity and a first number of sampling times (at step 1406). In some embodiments, the method includes recording, via the first air quantity analyzer, a curve of the relationship between the first air quantity and the first number of sampling times (at step 1408). According to some embodiments, the method includes determining, via the first air quantity analyzer, if a first tracer gas concentration has exceeded a threshold tracer gas concentration (at step 1410). The method may include sounding a first alarm in response to determining that the first tracer gas concentration has exceeded the threshold tracer gas concentration (at step 1412). In some embodiments, the method includes recording, via the first air quantity analyzer, the first tracer gas concentration in response to determining that the first tracer gas concentration has exceeded the threshold tracer gas concentration (at step 1414).

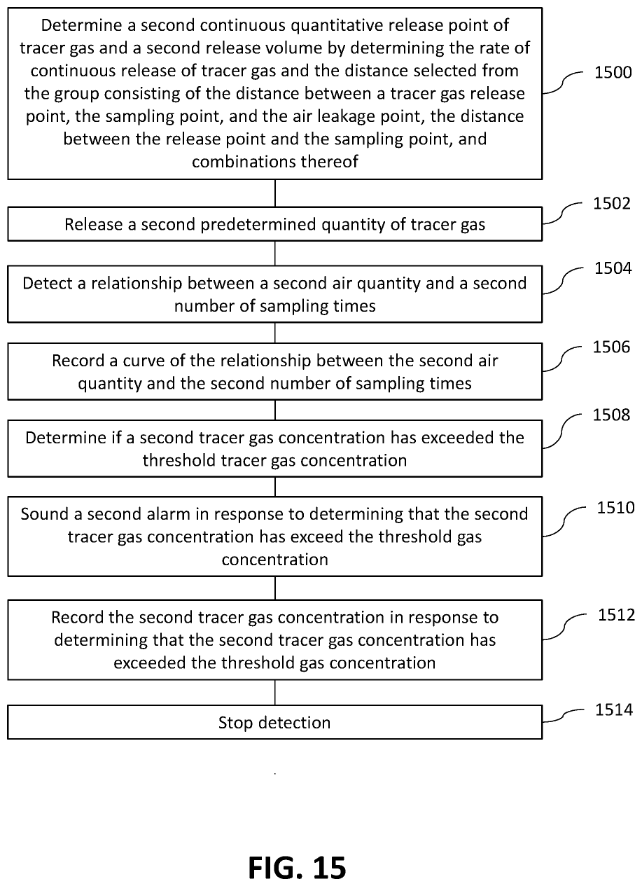

FIG. 15 illustrates a method of analyzing a second air quantity under local negative-pressure air leakage, according to some embodiments. According to some embodiments, the method includes determining a second continuous quantitative release point of tracer gas and a second release volume by determining the rate of continuous release of tracer gas and the distance selected from the group consisting of the distance between a tracer gas release point, the sampling point, and the air leakage point, the distance between the release point and the sampling point, and combinations thereof (at step 1500). The method may include releasing, via the releasing device, a second predetermined quantity of tracer gas (at step 1502). In some embodiments, the method includes detecting, via a second air quantity analyzer, a relationship between a second air quantity and a second number of sampling times (at step 1504). According to some embodiments, the method includes recording, via the second air quantity analyzer, the curve of the relationship between the second air quantity and the second number of sampling times (at step 1506). The method may include determining, via the second air quantity analyzer, if a second tracer gas concentration has exceeded the threshold tracer gas concentration (at step 1508). In some embodiments, the method includes sounding a second alarm in response to determining that the second tracer gas concentration has exceeded the threshold tracer gas concentration (at step 1510). According to some embodiments, the method includes recording, via the second air quantity analyzer, the second tracer gas concentration in response to determining that the second tracer gas concentration has exceeded the threshold tracer gas concentration (at step 1512). The method may include stopping detection (at step 1514).

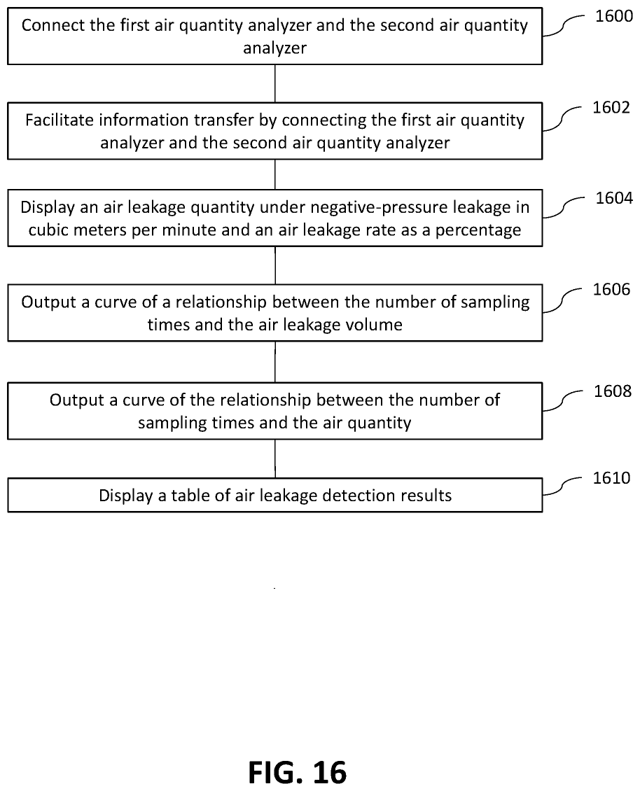

FIG. 16 illustrates a method of information transfer between air quantity analyzers and displaying information about local positive-pressure air leakage, according to some embodiments. In some embodiments, the method includes connecting, via the transmission module selected from the group consisting of the wired transmission module and the wireless transmission module, the first air quantity analyzer, the second air quantity analyzer, and combinations thereof (at step 1604). According to some embodiments, the method includes facilitating information transfer by connecting the first air quantity analyzer and the second air quantity analyzer (at step 1602). The method may include displaying, via the touch display, an air leakage quantity under negative-pressure leakage in cubic meters per minute and an air leakage rate as a percentage (at step 1604). In some embodiments, the method includes outputting, via the touch display, a curve of a relationship between the number of sampling times and the air leakage volume (at step 1606). According to some embodiments, the method includes outputting, via the touch display, a curve of a relationship between the number of sampling times and the air quantity (at step 1608). The method may include displaying, via a touch display, a table of air leakage detection results (at step 1610).

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section. To increase the clarity of various features, other features are not labeled in each figure.

The various features and processes described above may be used independently of one another or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, parallel, or some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless expressly stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless expressly stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only and are not intended to limit the scope of the inventions disclosed herein. Thus, nothing in the foregoing description implies that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. An air quantity analyzer for use in underground coal mines, comprising:
an explosion-proof enclosure;
a gas pipeline inside of the explosion-proof enclosure, the gas pipeline coupled to a mechanical component selected from the group consisting of an air sampler, a gas pool, and combinations thereof;
a detection device inside of the explosion-proof enclosure, the detection device having a vacuum pump, wherein the vacuum pump is coupled to the gas pipeline; and
a central processor inside of the explosion-proof enclosure and electrically coupled to the vacuum pump of the detection device, the central processor electrically coupled to an electric component selected from the group consisting of a light source, a detector, a power conversion module, a display and alarm module, a data communication module, and combinations thereof.

2. The air quantity analyzer for use in underground coal mines of claim 1, the air sampler further comprising:
a sampling head;
a current regulator coupled to the sampling head;
a dust and moisture removal device coupled to the current regulator; and
a spiral gas circuit coupled to the dust and moisture removal device.

3. The air quantity analyzer for use in underground coal mines of claim 2, wherein the central processor is electrically coupled to an electric component selected from the group consisting of a laser profiler, an airflow velocity sensor, and combinations thereof.

4. The air quantity analyzer for use in underground coal mines of claim 3, the power conversion module further comprising at least one rechargeable lithium battery arranged and configured to supply power to an electric component selected from the group consisting of the light source, the vacuum pump, the display and alarm module, the data communication module, the airflow velocity sensor, the laser profiler, and combinations thereof.

5. The air quantity analyzer for use in underground coal mines of claim 3, further comprising:
a second air quantity analyzer for use in underground coal mines, wherein the data communication module comprises a wired transmission module coupled to the second air quantity analyzer for use in underground coal mines, and
wherein the data communication module comprises a wireless transmission module coupled to the second air quantity analyzer for use in underground coal mines.

6. The air quantity analyzer for use in underground coal mines of claim 5, wherein the display and alarm module comprises a touch display, and wherein the display and alarm module is arranged and configured to provide an audible and visual alarm.

7. The air quantity analyzer for use in underground coal mines of claim 6, the explosion-proof enclosure further comprising:
a power indicator for displaying a current power level of the air quantity analyzer for use in underground coal mines;
a switch for turning the air quantity analyzer for use in underground coal mines off and on; and
a wired transmission module interface for interfacing with the wired transmission module.

8. The air quantity analyzer for use in underground coal mines of claim 2, the power conversion module further comprising at least one rechargeable lithium battery arranged and configured to supply power to an electric component selected from the group consisting of the light source, the vacuum pump, the display and alarm module, the data communication module, an airflow velocity sensor, a laser profiler, and combinations thereof.

9. The air quantity analyzer for use in underground coal mines of claim 2, further comprising:
a second air quantity analyzer for use in underground coal mines, wherein the data communication module comprises a wired transmission module coupled to the second air quantity analyzer for use in underground coal mines, and
wherein the data communication module comprises a wireless transmission module coupled to the second air quantity analyzer for use in underground coal mines.

10. The air quantity analyzer for use in underground coal mines of claim 9, wherein the display and alarm module comprises a touch display, and wherein the display and alarm module is arranged and configured to provide an audible and visual alarm.

11. The air quantity analyzer for use in underground coal mines of claim 10, the explosion-proof enclosure further comprising:
a power indicator for displaying a current power level of the air quantity analyzer for use in underground coal mines;
a switch for turning the air quantity analyzer for use in underground coal mines off and on; and
a wired transmission module interface for interfacing with the wired transmission module.

12. The air quantity analyzer for use in underground coal mines of claim 11, the power conversion module further comprising at least one rechargeable lithium battery arranged and configured to supply power to an electric component selected from the group consisting of the light source, the vacuum pump, the display and alarm module, the data communication module, an airflow velocity sensor, a laser profiler, and combinations thereof.

13. An air quantity analysis method for use in underground coal mines, comprising:
determining a rate of continuous release of tracer gas;
determining a distance selected from the group consisting of a distance between a tracer gas release point, a sampling point, and an air leakage point; a distance between the release point and the sampling point; and combinations thereof;
performing a test run of at least one air quantity analyzer;
ensuring, via performing the test run, that an air sampler is not blocked and a vacuum pump operates normally;
charging the at least one air quantity analyzer fully;
running the at least one air quantity analyzer at a selected placing point;
turning on a laser profiler and an airflow velocity sensor;
obtaining, via the laser profiler and the velocity sensor, an airflow velocity, a roadway cross-sectional area, and a shaft perimeter;
displaying, via a touch display, the rate of continuous release of tracer gas and the distance selected from the group consisting of the distance between a tracer gas release point, the sampling point, and the air leakage point; the distance between the release point and the sampling point; and
combinations thereof, and inputting, via the touch display, an interval time of a concentration sampling of tracer gas and a number of sampling times.

14. The air quantity analysis method for use in underground coal mines of claim 13, further comprising:
detecting a local positive-pressure air leakage;
determining a first continuous quantitative release point of tracer gas and a first release volume by determining the rate of continuous release of tracer gas and the distance selected from the group consisting of the distance between a tracer gas release point, the sampling point, and the air leakage point; the distance between the release point and the sampling point; and combinations thereof;
releasing, via a releasing device, a first predetermined quantity of tracer gas;
detecting, via a first air quantity analyzer, a relationship between a first air quantity and a first number of sampling times;
recording, via the first air quantity analyzer, a curve of the relationship between the first air quantity and the first number of sampling times;
determining, via the first air quantity analyzer, if a first tracer gas concentration has exceeded a threshold tracer gas concentration;
sounding a first alarm in response to determining that the first tracer gas concentration has exceeded the threshold tracer gas concentration; and
shutting down the first air quantity analyzer in response to determining that the first tracer gas concentration has exceeded the threshold tracer gas concentration.

15. The air quantity analysis method for use in underground coal mines of claim 14, further comprising:
determining a second continuous quantitative release point of tracer gas and a second release volume by determining the rate of continuous release of tracer gas and the distance selected from the group consisting of the distance between a tracer gas release point, the sampling point, and the air leakage point; the distance between the release point and the sampling point; and combinations thereof;
releasing, via the releasing device, a second predetermined quantity of tracer gas;
detecting, via a second air quantity analyzer, a relationship between a second air quantity and a second number of sampling times;
recording, via the second air quantity analyzer, the curve of the relationship between the second air quantity and the second number of sampling times;
determining, via the second air quantity analyzer, if a second tracer gas concentration has exceeded the threshold tracer gas concentration;
sounding a second alarm in response to determining that the second tracer gas concentration has exceeded the threshold tracer gas concentration; and
shutting down the second air quantity analyzer in response to determining that the second tracer gas concentration has exceeded the threshold tracer gas concentration.

16. The air quantity analysis method for use in underground coal mines of claim 15, further comprising:
displaying, via the touch display, an air leakage quantity under positive-pressure leakage in cubic meters per minute and an air leakage rate as a percentage;
outputting, via the touch display, a curve of a relationship between the number of sampling times and the air leakage volume;
outputting, via the touch display, a curve of a relationship between the number of sampling times and the air quantity; and
displaying, via the touch display, a table of air leakage detection results.

17. The air quantity analysis method for use in underground coal mines of claim 13, further comprising:
detecting a local negative-pressure air leakage;
determining a first continuous quantitative release point of tracer gas and a first release volume by determining the rate of continuous release of tracer gas and the distance selected from the group consisting of the distance between a tracer gas release point, the sampling point, and the air leakage point; the distance between the release point and the sampling point; and combinations thereof;
releasing, via a releasing device, a first predetermined quantity of tracer gas;
detecting, via a first air quantity analyzer, a relationship between a first air quantity and a first number of sampling times;
recording, via the first air quantity analyzer, a curve of the relationship between the first air quantity and the first number of sampling times;
determining, via the first air quantity analyzer, if a first tracer gas concentration has exceeded a threshold tracer gas concentration;
sounding a first alarm in response to determining that the first tracer gas concentration has exceeded the threshold tracer gas concentration; and
recording, via the first air quantity analyzer, the first tracer gas concentration in response to determining that the first tracer gas concentration has exceeded the threshold tracer gas concentration.

18. The air quantity analysis method for use in underground coal mines of claim 17, further comprising:
determining a second continuous quantitative release point of tracer gas and a second release volume by determining the rate of continuous release of tracer gas and the distance selected from the group consisting of the distance between a tracer gas release point, the sampling point, and the air leakage point; the distance between the release point and the sampling point; and combinations thereof;
releasing, via the releasing device, a second predetermined quantity of tracer gas;
detecting, via a second air quantity analyzer, a relationship between a second air quantity and a second number of sampling times;
recording, via the second air quantity analyzer, the curve of the relationship between the second air quantity and the second number of sampling times;
determining, via the second air quantity analyzer, if a second tracer gas concentration has exceeded the threshold tracer gas concentration;
sounding a second alarm in response to determining that the second tracer gas concentration has exceeded the threshold tracer gas concentration;
recording, via the second air quantity analyzer, the second tracer gas concentration in response to determining that the second tracer gas concentration has exceeded the threshold tracer gas concentration; and
stopping detection.

19. The air quantity analysis method for use in underground coal mines of claim 18, wherein each at least one air quantity analyzer further comprises a transmission module selected from the group consisting of a wired transmission module, a wireless transmission module, and combinations thereof, the method further comprising:
connecting, via the transmission module selected from the group consisting of the wired transmission module, the wireless transmission module, the first air quantity analyzer, the second air quantity analyzer, and combinations thereof; and
facilitating information transfer by connecting the first air quantity analyzer and the second air quantity analyzer.

20. The air quantity analysis method for use in underground coal mines of claim 18, further comprising:
displaying, via the touch display, an air leakage quantity under negative-pressure leakage in cubic meters per minute and an air leakage rate as a percentage;
outputting, via the touch display, a curve of a relationship between the number of sampling times and the air leakage volume;
outputting, via the touch display, a curve of a relationship between the number of sampling times and the air quantity; and
displaying, via a touch display, a table of air leakage detection results.
